(19) United States
(12) Patent Application Publication
(10) Pub. No.: US 2005/0137696 A1
(43) Pub. Date: Jun. 23, 2005

(54) Apparatus and methods for protecting against embolization during endovascular heart valve replacement

(75) Inventors: Amr Salahieh, Saratoga, CA (US); Brian D. Brandt, Santa Clara, CA (US); Dwight P. Morejohn, Davis, CA (US); Kenneth J. Michlitsch, Livermore, CA (US); Tom Saul, El Granada, CA (US)

Correspondence Address: WILSON SONSINI GOODRICH & ROSATI, 650 PAGE MILL ROAD, PALO ALTO, CA, 943041050, US

(73) Assignee: Sadra Medical, Campbell, CA (US)

(21) Appl. No.: 10/920,736

(22) Filed: Aug. 17, 2004

Related U.S. Application Data
(63) Continuation-in-part of application No. 10/746,280, filed on Dec. 23, 2003.

(51) Int. Cl. A61F 2/24

(52) U.S. Cl. 623/2.11

(57) ABSTRACT

Apparatus for protecting a patient against embolization during endovascular replacement of the patient's heart valve is provided, the apparatus including a replacement valve configured for endovascular delivery and deployment, and an embolic filter configured for disposal downstream of the replacement valve during deployment of the valve. Apparatus including a delivery catheter having an expandable replacement valve disposed therein, and an embolic filter advanceable along the delivery catheter for diverting emboli released during endovascular deployment of the replacement valve is also provided. Furthermore, methods for protecting a patient against embolization during endovascular replacement of the patient's heart valve are provided, the methods including the steps of endovascularly delivering a replacement valve to a vicinity of the patient's heart valve, endovascularly deploying an embolic filter downstream of the heart valve, and endovascularly deploying the replacement valve.

### REFERENCE TO RELATED APPLICATION

This application is a continuation-in-part application of Ser. No. 10/746,280, filed Dec. 23, 2003, which is incorporated herein by reference in its entirety and to which application we claim priority under 35 USC § 120.

### BACKGROUND OF THE INVENTION

The present invention relates to methods and apparatus for protecting a patient from embolization during endovascular replacement of the patient's heart valve. More particularly, the present invention relates to methods and apparatus for providing embolic protection by filtering blood downstream of the valve during endovascular replacement.

Heart valve surgery is used to repair or replace diseased heart valves. Valve surgery typically is an open-heart procedure conducted under general anesthesia. An incision is made through a patient's sternum (sternotomy), and the patient's heart is stopped while blood flow is rerouted through a heart-lung bypass machine. The valve then is surgically repaired or replaced, blood is rerouted back through the patient's heart, the heart is restarted, and the patient is sewn up.

Valve replacement may be indicated when there is a narrowing of the native heart valve, commonly referred to as stenosis, or when the native valve leaks or regurgitates. When replacing the valve, the native valve is excised and replaced with either a biologic or a mechanical valve. Mechanical valves require lifelong anticoagulant medication to prevent blood clot formation, and clicking of the valve often may be heard through the chest. Biologic tissue valves typically do not require such medication. Tissue valves may be obtained from cadavers or may be porcine or bovine, and are commonly attached to synthetic rings that are secured to the patient's heart.

Valve replacement surgery is a highly invasive operation with significant concomitant risk. Risks include bleeding, infection, stroke, heart attack, arrhythmia, renal failure, adverse reactions to the anesthesia medications, as well as sudden death. 2-5% of patients die during surgery.

Post-surgery, patients temporarily may be confused due to emboli and other factors associated with the heart-lung machine. The first 2-3 days following surgery are spent in an intensive care unit where heart functions can be closely monitored. The average hospital stay is between 1 to 2 weeks, with several more weeks to months required for complete recovery.

In recent years, advancements in minimally invasive surgery and interventional cardiology have encouraged some investigators to pursue percutaneous, endovascular replacement of the aortic heart valve. See, e.g., U.S. Pat. No. 6,168,614, which is incorporated herein by reference in its entirety. The replacement valve may be deployed across the native diseased valve to permanently hold the native valve open, thereby alleviating a need to excise the native valve and to position the replacement valve in place of the native valve. Optionally, a valvuloplasty may be performed prior to, or after, deployment of the replacement valve.

Since the native valve may be calcified or stenosed, valvuloplasty and/or deployment of the replacement valve poses a risk of loosening and releasing embolic material into the patient's blood stream. This material may, for example, travel downstream through the patient's aorta and carotids to the cerebral vasculature of the brain. Thus, a risk exists of reduction in mental faculties, stroke or even death during endovascular heart valve replacement, due to release of embolic material.

In view of the foregoing, it would be desirable to provide methods and apparatus for protecting against embolization during endovascular replacement of a patient's heart valve.

### SUMMARY OF THE INVENTION

One aspect of the invention provides apparatus for protecting against embolization during endovascularly replacement of a patient's heart valve, including: a replacement valve configured for endovascular delivery and deployment; and an embolic filter configured for disposal downstream of the replacement valve during endovascular deployment of the valve.

Another aspect of the invention provides a method for protecting a patient against embolization during endovascular replacement of the patient's heart valve, including the steps of: endovascularly delivering a replacement valve to a vicinity of the patient's heart valve; endovascularly deploying an embolic filter downstream of the heart valve; and endovascularly deploying the replacement valve. The method may also include the step removing the embolic filter from the patient after endovascular deployment of the replacement valve. In embodiments in which the heart valve is an aortic valve, the endovascular delivery step may include the step of endovasculary delivering the replacement valve along a retrograde approach, and the filter deployment step may include deploying the filter in the patient's aorta. The method may also include the step of endovascularly delivering an expandable balloon to the vicinity of the heart valve and performing valvuloplasty with the expandable balloon.

Yet another aspect of the invention provides apparatus for protecting against embolization during endovascularly replacement of a patient's heart valve, including: a delivery catheter having an expandable replacement valve disposed therein; and an embolic filter advanceable along the delivery catheter for diverting emboli released during endovascular deployment of the replacement valve.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-F are side views, partially in section, illustrating a method and apparatus for protecting a patient against embolization during endovascular replacement of the patient's diseased aortic valve.

FIG. 2 is a side view, partially in section, illustrating an alternative embodiment of the apparatus and method of FIGS. 1.

FIGS. 3A-D are schematic side-sectional views illustrating another alternative method and apparatus for protecting against embolization during endovascular valve replacement.

FIGS. 4A-D are side-views, partially in section, illustrating yet another method and apparatus for protecting against embolization, wherein an embolic filter is coaxially advanced over, or coupled to, an exterior of a replacement valve delivery catheter.

FIGS. 5A-F are schematic isometric views illustrating alternative embodiments of the apparatus of FIGS. 4.

FIGS. 6A-D are side views, partially in section, illustrating another method and apparatus for protecting against embolization.

FIG. 7A-B are cross- and side-sectional detail views, respectively, along section lines A-A and B-B of FIG. 6A, respectively, illustrating an optional method and apparatus for enhancing blood flow to the patient's coronary arteries while utilizing the apparatus of FIGS. 6.

FIG. 8 is a schematic view of an embodiment of the apparatus of FIGS. 6 comprising a measuring element.

FIGS. 9A-I are schematic views of exemplary alternative embodiments of the apparatus of FIGS. 6.

FIGS. 10A-B are detail schematic views illustrating a spiral wound support structure.

FIG. 11 is a detail schematic view illustrating longitudinal supports for maintaining a length of the apparatus.

FIGS. 12A-C are detail schematic views illustrating alternative deployment and retrieval methods for the apparatus.

FIGS. 13A-G are schematic views and side views, partially in section, illustrating a method and apparatus for protecting a patient against embolization during endovascular valvuloplasty and replacement of the patient's diseased aortic valve.

### DETAILED DESCRIPTION OF THE INVENTION

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to methods and apparatus for protecting a patient against embolization during endovascular replacement of the patient's diseased heart valve. More particularly, the present invention relates to methods and apparatus for providing embolic protection by filtering blood downstream of the valve during endovascular replacement. Applicant has previously described methods and apparatus for endovascularly replacing a patient's diseased heart valve, for example, in co-pending U.S. patent application Ser. No. 10/746,280, filed Dec. 23, 2003, from which the present application claims priority and which previously has been incorporated herein by reference.

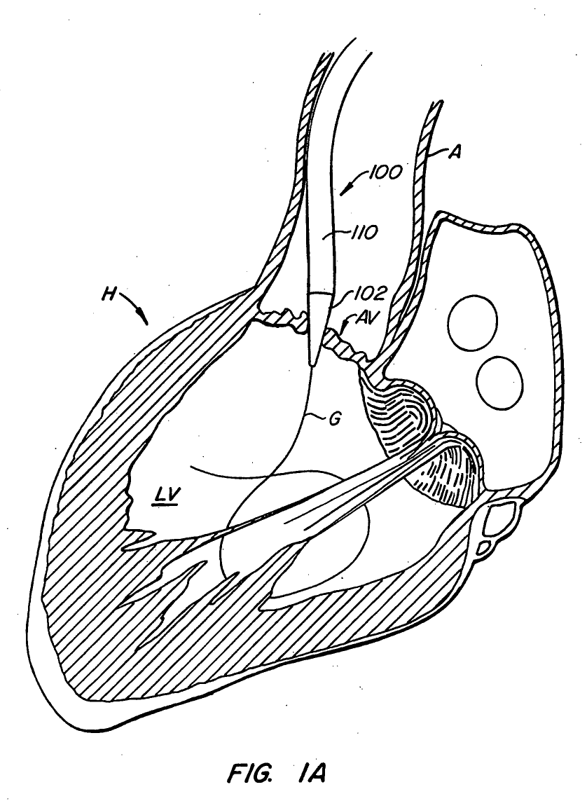

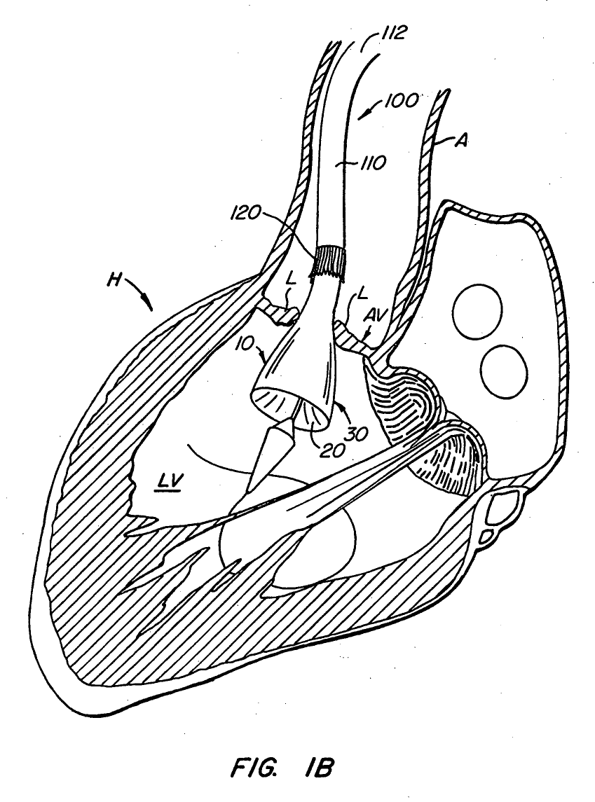

Referring now to FIGS. 1, a first embodiment of a method and apparatus for protecting a patient against embolization during endovascular replacement of the patient's diseased aortic valve is described. In FIGS. 1, replacement valve apparatus 10 illustratively comprises replacement valve 20 disposed within and coupled to expandable anchor 30. Apparatus 10 is provided only for the sake of illustration, and any other replacement valve apparatus may alternatively be provided.

Replacement valve 20 preferably is from biologic tissues, e.g. porcine valve leaflets or bovine or equine pericardium tissues. Alternatively, it can be made from tissue-engineered materials (such as extracellular matrix material from Small Intestinal Submucosa (SIS)). As yet another alternative, the replacement valve may be prosthetic from an elastomeric polymer or silicone, or a Nitinol or stainless steel mesh or pattern (sputtered, chemically milled or laser cut). Replacement valve 20 may comprise leaflets that may also be made of a composite of the elastomeric or silicone materials and metal alloys or other fibers, such Kevlar or carbon. Anchor 30 may, for example, dynamically self-expand; expand via a hydraulic or pneumatic force, such as expansion of a balloon catheter therein; expand via a non-hydraulic or non-pneumatic force; and/or be foreshortened in order to increase its radial strength.

Replacement valve apparatus 10 is reversibly coupled to delivery system 100, which illustratively comprises sheath 110 having lumen 112, as well as control wires 50 and control rods or tubes 60. Delivery system 100 may further comprise leaflet engagement element 120, as well as filter structure 61A. Engagement element 120, which may be releasably coupled to the anchor, is disposed between the anchor and tubes 60 of the delivery system. Filter structure 61A may, for example, comprise a membrane or braid, e.g., an expandable Nitinol braid, circumferentially disposed about tubes 60. Structure 61A preferably comprises a specified porosity, for example, preferably comprises a plurality of pores on the order of about 100 μm or less to facilitate blood flow therethrough while filtering dangerously sized emboli from the blood. Structure 61A may be used independently or in combination with engagement element 120 to provide embolic protection during deployment of replacement valve apparatus 10.

Replacement valve apparatus 10 is configured for disposal in a delivery configuration within lumen 112 of sheath 110 to facilitate percutaneous, endoluminal delivery thereof. Wires 50, tubes 60, element 120 and/or sheath 110 of delivery system 100 may be utilized to deploy apparatus 10 from the delivery configuration to an expanded deployed configuration.

In FIG. 1A, sheath 110 of delivery system 100, having apparatus 10 disposed therein, may be endovascularly advanced over guide wire G, preferably in a retrograde fashion (although an antegrade or hybrid approach alternatively may be used), through a patient's aorta A to the patient's diseased aortic valve AV. A nosecone 102 precedes sheath 110 in a known manner. In FIG. 1B, sheath 110 is positioned such that its distal region is disposed within left ventricle LV of the patient's heart H.

After properly aligning the apparatus relative to anatomical landmarks, such as the patient's coronary ostia or the patient's native valve leaflets L, apparatus 10 may be deployed from lumen 112 of sheath 110, for example, under fluoroscopic guidance. Anchor 30 of apparatus 10 illustratively self-expands to a partially deployed configuration, as in FIG. 1C. Leaflet engagement element 120 of delivery system 100 preferably self-expands along with anchor 30.

Element 120 initially is deployed proximal of the patient's native valve leaflets L, such that the element sealingly engages against the patient's aorta A to capture or otherwise filter emboli E that may be released during maneuvering or deployment of apparatus 10. Element 120 may also direct emboli E into filter structure 61A and out through sheath 110, such that the emboli do not travel downstream through the patient's aorta or into the patient's cerebral vasculature. Suction optionally may be drawn through lumen 112 of sheath 110 during placement of apparatus 10 to facilitate aspiration or removal of emboli E from the patient's blood stream to further reduce a risk of embolization.

As seen in FIG. 1D, apparatus 10 and element 120 may be advanced, and/or anchor 30 may be foreshortened, until the engagement element positively registers against valve leaflets L, thereby ensuring proper positioning of apparatus 10. Upon positive registration of element 120 against leaflets L, element 120 precludes further distal migration of apparatus 10 during additional foreshortening or other deployment of apparatus 10, thereby reducing a risk of improperly positioning the apparatus. Once expanded to the fully deployed configuration of FIG. 1D, replacement valve apparatus 10 regulates normal blood flow between left ventricle LV and aorta A.

As discussed, emboli can be generated during manipulation and placement of apparatus 10, e.g., from the diseased native leaflets or from surrounding aortic tissue. Arrows 61B in FIG. 1E show blood flowing past engagement element 120 and through porous filter structure 61A. While blood is able to flow through the filter structure, emboli E are trapped in the delivery system and removed with it at the end of the procedure or aspirated via suction during the procedure. FIG. 1E also details engagement of element 120 against the native leaflets and illustrates locks 40, which optionally may be used to maintain apparatus 10 in the fully deployed configuration.

As seen in FIG. 1F, delivery system 100 may be decoupled from apparatus 10 and removed from the patient, thereby removing the embolic filter provided by element 120 and filter structure 61A, and completing protected, beating-heart, endovascular replacement of the patient's diseased aortic valve.

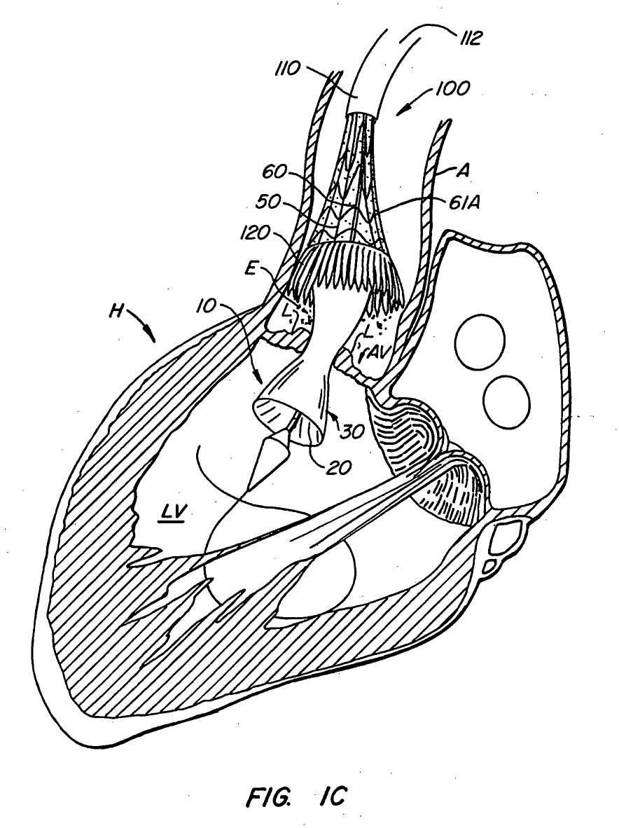

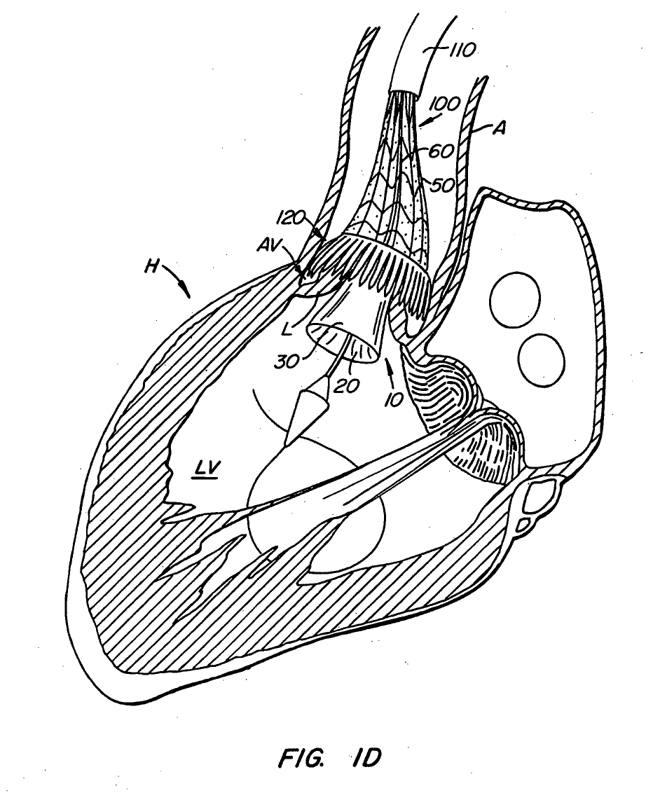

With reference to FIG. 2, an alternative embodiment of the apparatus of FIGS. 1 is described, wherein leaflet engagement element 120 is coupled to anchor 30 of apparatus 10, rather than to delivery system 100. Engagement element 120 remains implanted in the patient post-deployment of apparatus 10, and leaflets L of native aortic valve AV are sandwiched between the engagement element and anchor 30. In this manner, element 120 positively registers apparatus 10 relative to the leaflets and precludes distal migration of the apparatus over time. Furthermore, since element 120 may act as an embolic filter during deployment of apparatus 10, any emboli E captured against element 120 may harmlessly remain sandwiched between the element and the patient's native leaflets, thereby reducing a risk of embolization.

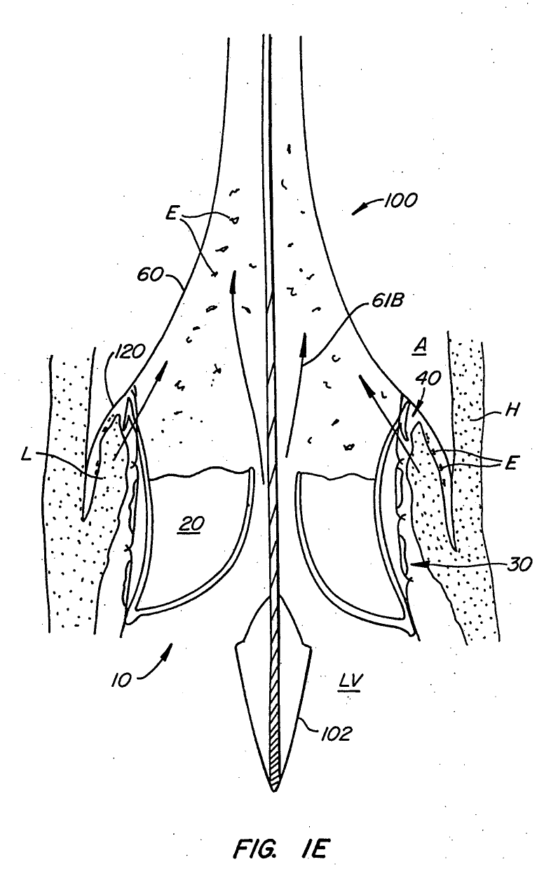

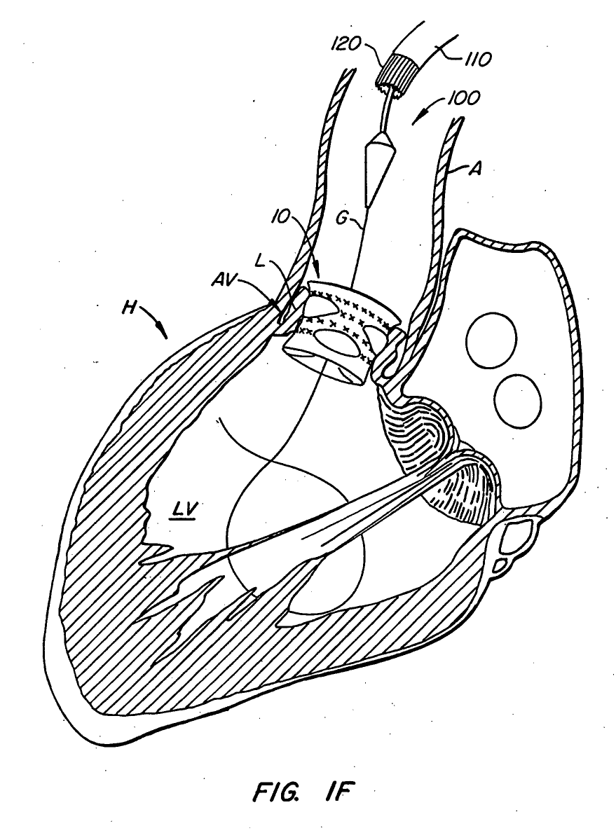

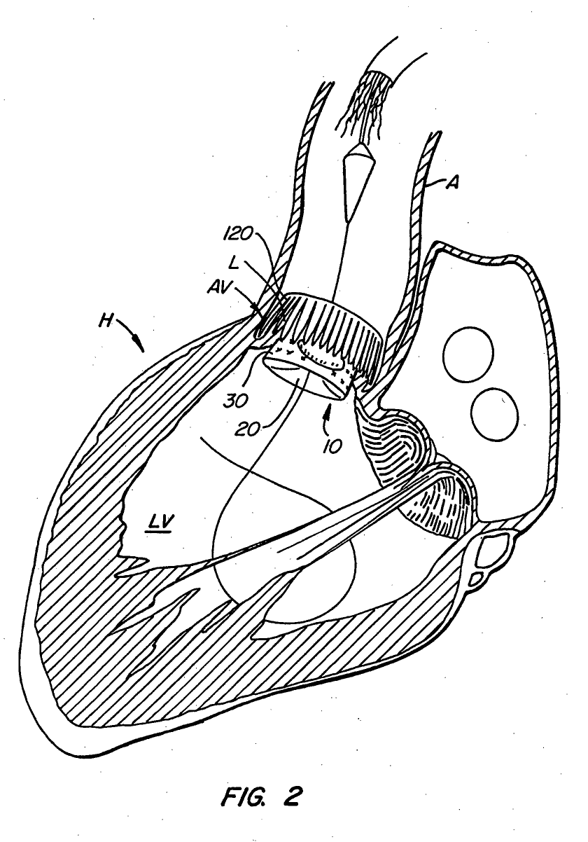

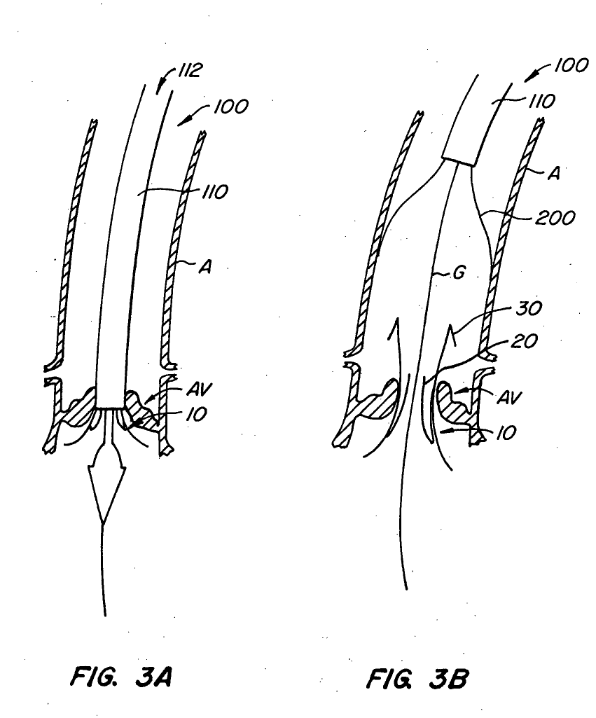

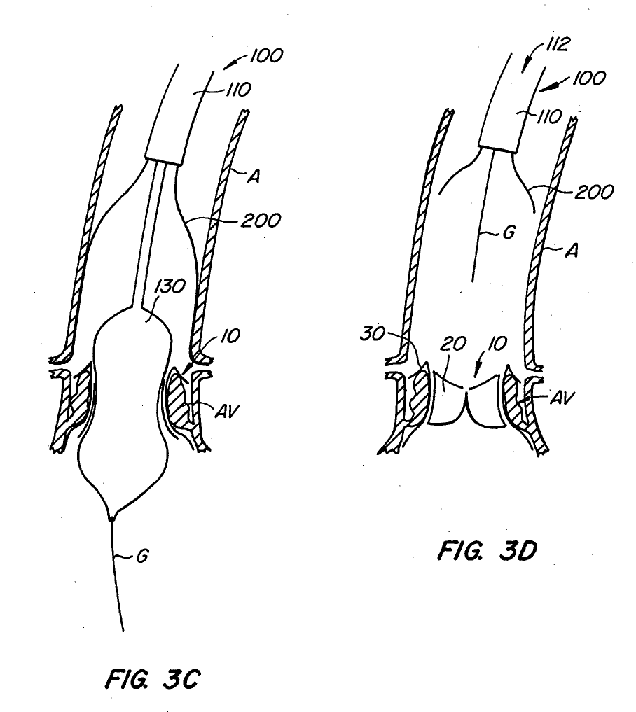

Referring now to FIGS. 3, another alternative method and apparatus for protecting against embolization is described. In FIG. 3A, replacement valve apparatus 10 is once again disposed within lumen 112 of sheath 110 of delivery system 100. As seen in FIG. 3B, the apparatus is deployed from the lumen and expands to a partially deployed configuration across the patient's native aortic valve AV. A separate, expandable embolic filter 200 is also deployed from lumen 112 downstream of apparatus 10 within the patient's aorta A, such that the filter sealingly engages the aorta. Any emboli generated during further expansion of apparatus 10 to a fully deployed configuration would be filtered out of the patient's blood stream via the filter and/or lumen 112 of sheath 110. Filter 200 preferably is porous to allow for uninterrupted blood flow through aorta A during use of the filter. The filter may, for example, be fabricated from a porous polymer membrane, or from a braid or mesh, e.g. a braided Nitinol structure.

As seen in FIG. 3C, balloon catheter 130 may be advanced through sheath 110 and filter 200 into apparatus 10. The balloon may be inflated to further expand apparatus 10 to the fully deployed configuration. Emboli E generated during deployment of apparatus 10 then may be captured or otherwise filtered by filter 200. As seen in FIG. 3D, balloon catheter 130 then may be deflated and removed from the patient, filter 200 may be collapsed within lumen 112 of sheath 110, and delivery system 100 may be removed, thereby completing the protected valve replacement procedure.

It should be understood that balloon catheter 130 alternatively may be used to perform valvuloplasty prior to placement of apparatus 10 across the diseased valve. In this configuration, filter 200 may be utilized to capture emboli generated during the valvuloplasty procedure and prior to placement of apparatus 10, as well as to provide embolic protection during placement and deployment of the replacement valve apparatus. After the valvuloplasty procedure, apparatus 10 may be deployed with or without balloon catheter 130.

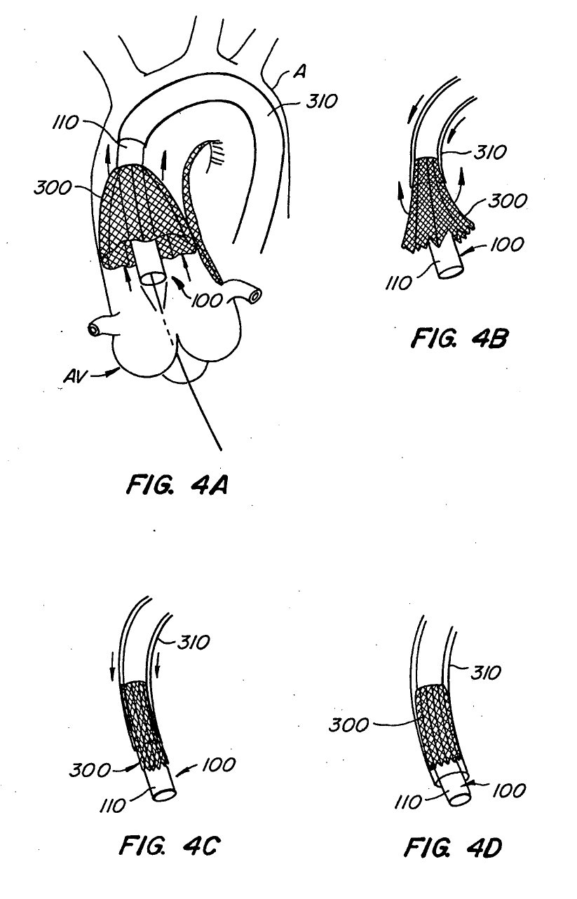

Referring now to FIGS. 4, yet another method and apparatus for protecting against embolization is described, wherein an embolic filter is coaxially advanced over, or is coupled to, an exterior of a replacement valve delivery catheter. In FIG. 4A, replacement valve apparatus, e.g., apparatus 10, is disposed for delivery within the lumen of a delivery sheath, e.g., delivery sheath 110 of delivery system 100. Expandable embolic filter 300 is either coupled to, or is advanceable over, an exterior surface of the delivery sheath.

When filter 300 is advanceable over the delivery sheath, sheath 110 may be positioned in a vicinity of a patient's diseased heart valve, as shown, and filter 300 may be advanced along the exterior of delivery sheath via coaxially-disposed pusher sheath 310. Delivery sheath 110 preferably comprises a motion limitation element, such as a cross-section of locally increased diameter (not shown), which limits advancement of filter 300 relative to the delivery sheath.

When filter 300 is coupled to the exterior of delivery sheath 110, the filter may be collapsed for delivery by advancing pusher sheath 310 over the filter, such that the filter is sandwiched in an annular space between delivery sheath 110 and pusher sheath 310. Replacement valve apparatus 10, delivery system 100, filter 300 and pusher sheath 310 then may be endovascularly advanced to the vicinity of the patient's diseased heart valve AV. Once properly positioned, the pusher sheath may be retracted, such that filter 300 dynamically expands into sealing contact with the patient's aorta A, as in FIG. 4A.

Regardless of whether filter 300 is coupled to, or is advanceable over, delivery sheath 110; once properly positioned, the filter sealingly contacts the patient's aorta and filters blood passing through the aorta to remove any harmful emboli (arrows illustrate blood flow in FIG. 4A). Thus, the replacement valve apparatus may be deployed while the filter protects against embolization. As seen in FIG. 4B, once embolic protection is no longer desired, e.g., after endovascular replacement of the patient's diseased heart valve, filter 300 may be collapsed for removal by advancing pusher sheath 310 relative to delivery sheath 110 and filter 300. FIG. 4B illustrates the filter after partial collapse, while FIG. 4C shows the filter nearly completely collapsed. In FIG. 4D, filter 300 is fully enclosed within the annular space between delivery sheath 110 and pusher sheath 310. Any dangerous emboli generated during deployment of the replacement valve apparatus are trapped between filter 300 and the exterior surface of delivery sheath 110. Delivery system 100, filter 300 and pusher sheath 310 then may be removed from the patient to complete the procedure.

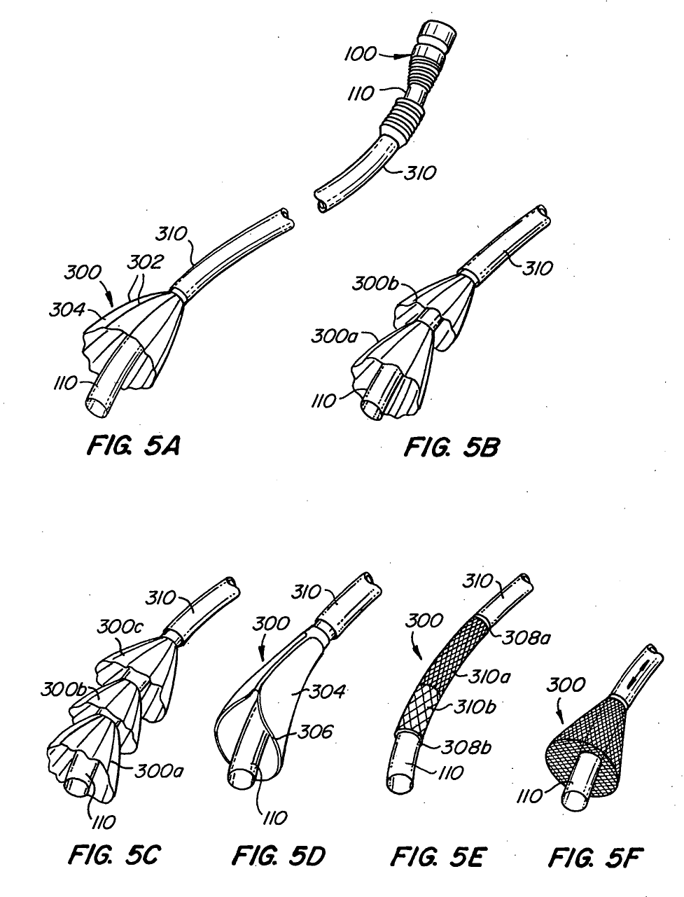

With reference to FIGS. 5, alternative embodiments of the embolic protection apparatus of FIGS. 4 are described. In FIG. 5A, filter 300 is substantially the same as in FIGS. 4, but a proximal control region of the embolic protection apparatus, which is disposed outside of the patient, is also described. Region 400, which generally is shown as useable with any of the embodiments of FIG. 5, comprises proximal handle 115 of delivery sheath 110, as well as proximal handle 315 of pusher sheath 310. A medical practitioner may grasp handle 115 with a first hand and handle 315 with a second hand for relative movement of pusher sheath 310 and delivery sheath 110.

In FIG. 5B, filter 300 comprises first filter 300a and second filter 300b. As with the unitary filter of FIGS. 4 and 5A, filters 300a and 300b may be coupled to, or advanceable over, the exterior of sheath 110. As another alternative, filter 300a may be coupled to the delivery sheath, while filter 300b is advanceable over the sheath. Filters 300a and 300b may be deployed and retrieved as described previously with respect to FIGS. 4. Specifically, one or both of the filters may be advanced along delivery sheath 110 via pusher sheath 310, or may be expanded from the annular space between the delivery and pusher sheaths. Likewise, the filters may be collapsed for retrieval within the annular space.

Providing multiple filters may reduce a risk of embolization via emboli inadvertently bypassing the first filter, for example, due to an imperfect seal between the filter and the patient's anatomy. Additionally, each of the filters may have a different porosity; for example, filter 300a may provide a rough filter to remove larger emboli, while filter 300b may comprise a finer porosity to capture smaller emboli. Filtering the emboli through multiple filters may spread the emboli over multiple filters, thereby reducing a risk of impeding blood flow due to clogging of a single filter with too many emboli. The embodiment of FIG. 5C extends these concepts: filter 300 comprises first filter 300a, second filter 300b and third filter 300c. As will be apparent, any number of filters may be provided.

The filters of FIGS. 5A-5C generally comprise expandable baskets having self-expanding ribs 302, e.g., Nitinol or spring steel ribs, surrounded by a porous and/or permeable filter membrane 304. FIG. 5D provides an alternative filter 300 comprising a self-expanding wire loop 306 surrounded by membrane 304. Deployment and retrieval of filter 300 of FIG. 5D is similar to that of filters 300 of FIGS. 5A-5C.

FIGS. 5E and 5F illustrate yet another embodiment of filter 300. In FIG. 5E, filter 300 is shown in a collapsed delivery configuration against the exterior surface of delivery sheath 110. Filter 300 is proximally coupled to pusher sheath 310 at attachment point 308a, and is distally coupled to, or motion limited by, delivery sheath 110 at attachment point 308b. Filter 300 comprises proximal braid 310a and distal braid 310b, e.g., proximal and distal Nitinol braids. The proximal braid preferably comprises a tighter weave for filtering smaller emboli, and may also be covered by a permeable/porous membrane (not shown). Distal braid 310b comprises a more open braid to facilitate expansion, as well as capture of larger emboli.

In FIG. 5F, pusher sheath 310 has been advanced relative to delivery sheath 110, thereby expanding filter 300 for capturing emboli. Once embolic protection is no longer desired, e.g., after endovascular replacement of the patient's diseased heart valve, pusher sheath 310 may be retracted relative to the delivery sheath, which collapses the filter back to the delivery configuration of FIG. 5E and captures emboli between the filter and the delivery sheath. As another alternative, pusher sheath 310 may be further advanced relative to the delivery sheath, thereby collapsing the filter into a retrieval configuration wherein the proximal braid covers the distal braid (not shown).

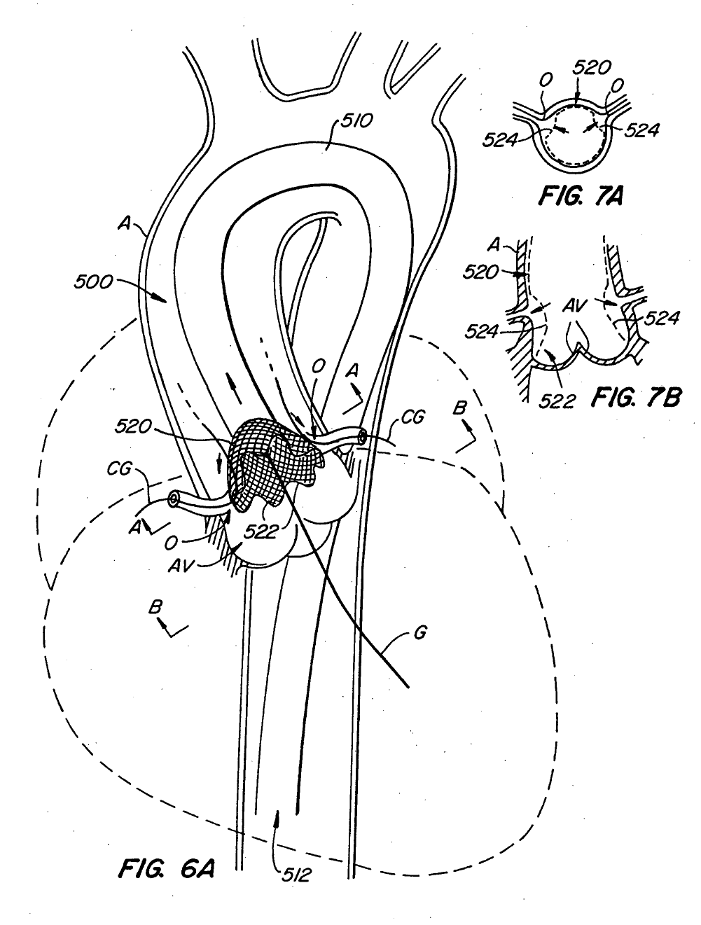

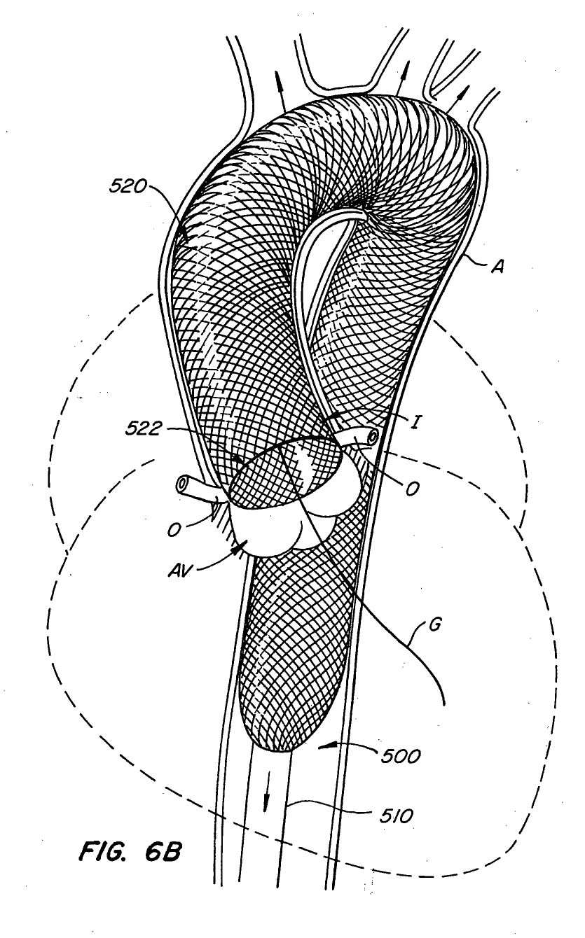

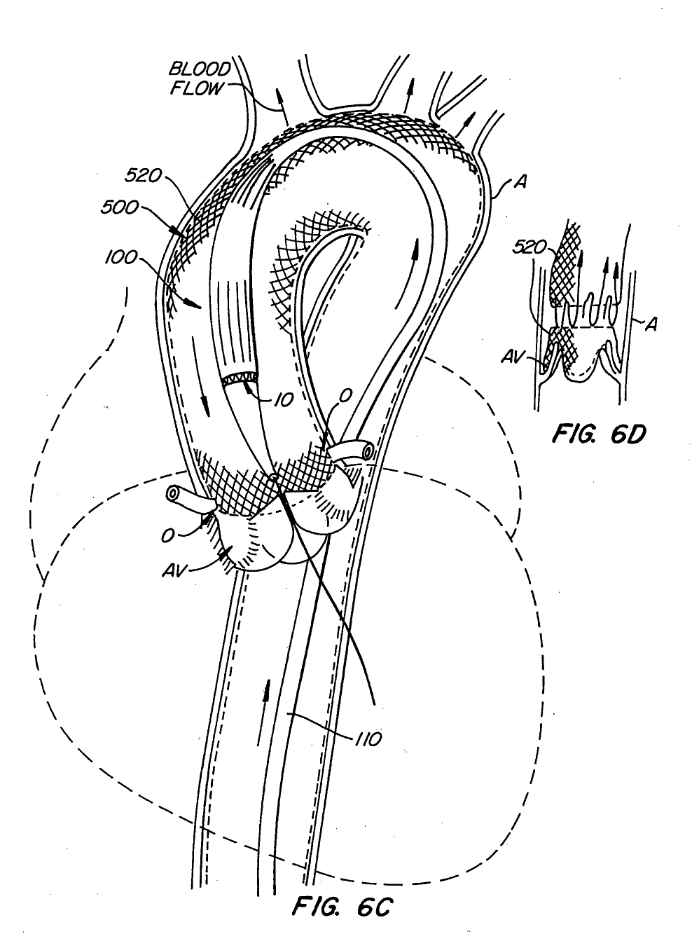

Referring now to FIGS. 6, another method and apparatus for protecting against embolization is described. In FIG. 6A, guidewire G has been percutaneously advanced through a patient's aorta A, past the patient's diseased aortic valve AV and into the left ventricle. Coronary guidewires CG may also be provided to facilitate proper positioning of elements advanced over guidewire G.

Embolic protection system 500 has been endovascularly advanced over guidewire G to the vicinity of the patient's aortic valve AV. System 500 includes exterior sheath 510 and embolic filter 520. The embolic filter may be collapsed for delivery and/or retrieval within lumen 512 of the sheath. As seen in FIGS. 6A and 6B, exterior sheath 510 may be withdrawn relative to filter 520, such that the filter self-expands into contact with the patient's anatomy. The open mesh of the braid, e.g. Nitinol braid, from which the filter is fabricated, provides filtered perfusion: filtered blood continues to flow through the filter and through the patient's aorta, as well as through side-branchings off of the aorta. Optionally, filter 520 may also comprise a permeable/porous membrane to assist filtering.

As shown in FIG. 6A, filter 520 optionally may comprise a scalloped distal edge 522 that fits behind the valve leaflets and over the leaflet commissures of aortic valve AV. The depth, number and/or shape(s) of distal edge 522 may be specified, as desired. Furthermore, marking indicia I (see FIG. 6B) may be provided on or near the edge to facilitate proper alignment of the edge with the patient's coronary ostia O. FIG. 6B illustrates an alternative embodiment of the filter wherein distal edge 522 is substantially planar. This may simplify placement of the filter without requiring complicated alignment with the patient's coronary ostia O, and the planar distal edge may simply rest on or near the valve leaflet commissures.

In addition to providing embolic protection, filter 520 may aid delivery of replacement valve apparatus. As seen in FIG. 6B, filter 520 contacts the inner wall of aorta A over a significant distance, thereby providing a non-slip protective layer for guiding additional catheters past blood vessel branches without damaging the vessel walls. As seen in the cutaway view of FIG. 6C, delivery system 100, having replacement valve apparatus 10 disposed therein, may then be advanced through embolic protection system 500; and endovascular, beating-heart replacement of the patient's diseased aortic valve AV may proceed in an embolically protected manner. As will be apparent, any alternative replacement valve apparatus and delivery system may be used in combination with embolic protection system 500. Furthermore, as seen in the detail view of FIG. 6D, all or part of filter 520 may be detachable and remain as part of the implanted replacement valve apparatus, e.g., as an anchor for the replacement valve.

Referring now to FIGS. 7, optional end geometry for filter 520 is described. As seen in FIG. 7B, distal edge 522 of filter 520 may distally extend into the cusps of the patient's diseased valve, for example, as a means to reference distances and/or to ensure full engagement. In order to guarantee adequate blood flow to the patient's coronary arteries, filter 520 may comprise heat-set or otherwise-formed indentations 524 that increase surface area flow through the filter to the patient;s coronary arteries. The indentations may also aid proper alignment of the replacement valve apparatus, e.g., may be used in conjunction with coronary guidewires CG.

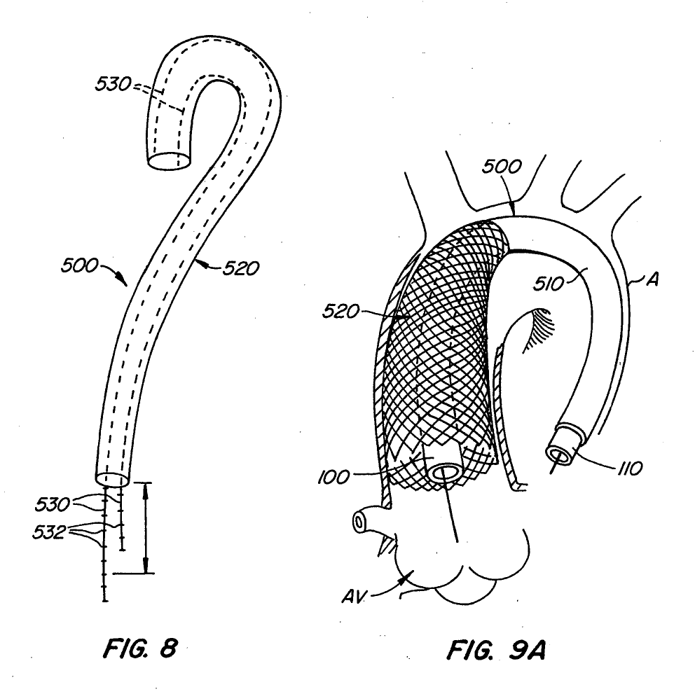

With reference to FIG. 8, an embodiment of embolic protection apparatus 500 is described comprising a measuring element. Embolic filter 520 may, for example, comprise a pair of opposed thin wires 530 that are anchored to the distal end of the filter and extend out the other end to provide a measuring element. The wires optionally may be radiopaque to facilitate visualization. Wires 530 comprise measurement indicia 532 on their proximal ends that give the distance between the indicia and the distal end of the wire. The average distance measured between the two wires provides the center axis distance through the patient's aorta to the valve commissures.

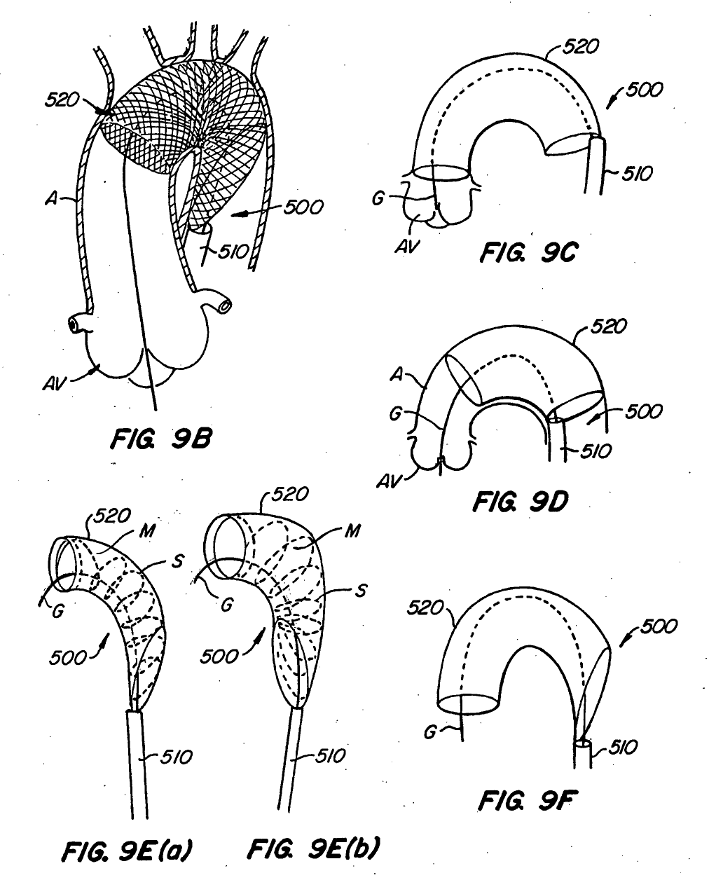

Referring now to FIGS. 9, various exemplary alternative embodiments of embolic protection system 500 are described. In FIG. 9A, a shorter version of embolic filter 520 is shown. The filter is disposed in the annular space between exterior sheath 510 and delivery system 100/replacement valve apparatus 10. The filter may be fabricated in a shorter length, or may be only partially deployed to a desired length.

FIG. 9B illustrates another optionally short-necked version of filter 520. However, unlike the filter of FIG. 9A, the proximal end of filter 520 in FIG. 9B is at least partially disconnected from sheath 510. Thus, filter 520 is a diverter that diverts emboli past the primary upper circulatory branchings of aorta A, e.g., those leading to the patient's carotid arteries, thereby protecting the patient from cerebral embolization. The emboli then may be allowed to continue downstream to less critical and/or dangerous regions of the patient's anatomy.

Optionally, suction may be applied through the lumen of sheath 510 to remove at least a portion of the emboli from the patient. Alternatively, a stand-alone suction catheter (not shown) may be advanced over, through or alongside sheath 510 to the vicinity of, or within, filter 520; suction then may be drawn through the suction catheter to aspirate the emboli. The suction catheter optionally may be part of delivery system 100, e.g., sheath 110.

The proximal end of filter 520 illustratively comprises a tapered or angled opening to facilitate collapse and removal of the filter from the patient. The distal end of the filter may likewise be tapered or angled in any desired direction or configuration.

In FIG. 9B, replacement valve apparatus optionally may be deployed directly through sheath 510 without an intervening delivery sheath. Alternatively, a delivery sheath, such as sheath 110, may be provided, as described previously. The delivery sheath may be advanced through or adjacent to filter sheath 510; alternatively, sheath 510 may be removed during placement of the replacement valve apparatus.

FIG. 9C illustrates an alternative embodiment of filter 520 wherein the filter comprises a permeable or porous membrane, web, film, etc., as opposed to a braid. The membrane may comprise a specified porosity, for example, pores of about 100 μm or less. In FIG. 9C, the proximal opening of filter 520 has been squared off. FIG. 9D illustrates an embodiment wherein sheath 510 is disposed along the opposing side of the patient's aorta A, as compared to the embodiment of FIG. 9C.

In FIG. 9E, filter 520 comprises membrane M with reinforcing, spiral-wound support S. The support optionally may be disposed within a guide track of the membrane and may be advanced or retracted within the membrane, as desired. FIG. 9E illustratively shows the proximal end of filter 520 tapered or angled in two different configurations; in FIG. 9E(a), the taper distally extends towards the lesser curvature of the aorta, while in FIG. 9E(b), the taper distally extends towards the greater curvature. Additional configurations will be apparent.

FIG. 9F illustrates a membrane embodiment of filter 520, which is similar to the braid embodiment of FIG. 9B. FIG. 9G illustrates another membrane/spiral-wound embodiment of filter 520. However, the filter of FIG. 9G is proximally attached to sheath 510, such that embolic particles are captured and removed from the patient, rather than diverted. FIG. 9H provides another proximally attached embodiment of the filter having one or more regions of specially designed porosity P. For example, the size and/or density of the pores may be varied as desired in the vicinity of vessel branchings, e.g., to enhance blood flow and/or to more finely filter particles.

Filter 520 may have a biased profile, e.g., such that it naturally assumes the curve of the patient's aorta. Alternatively, the filter may comprise a non-biased or straight profile as in FIG. 9I, which may be urged into a curved configuration. In FIG. 9I, filter 520 comprises membrane M strung between longitudinal support structure S.

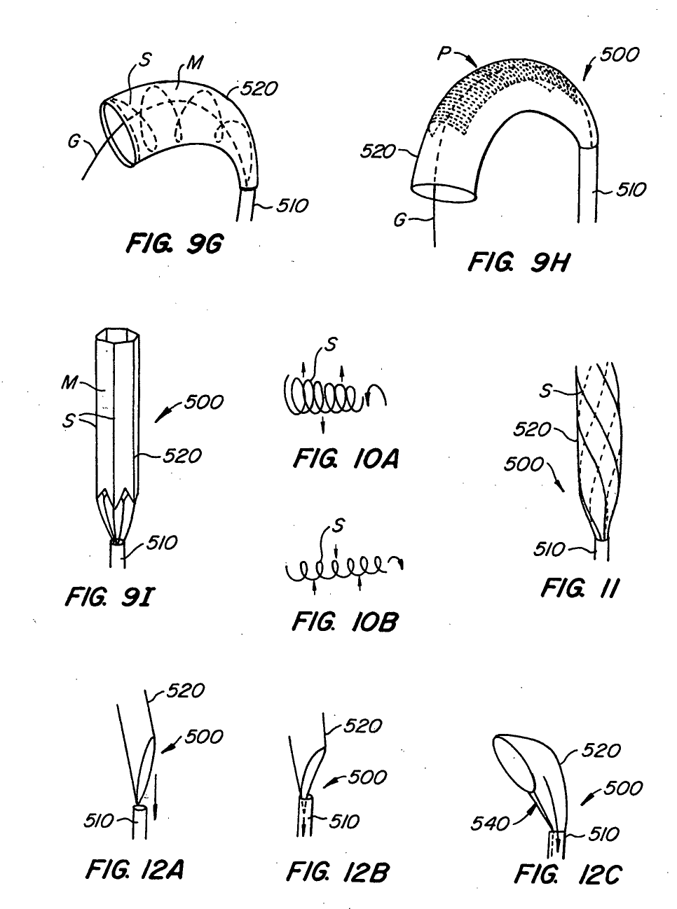

Referring now to FIGS. 10, a spiral wound structure for use with any of the previously described filters is described. Structure S acts as a radially-expansive support when torqued in a first direction, as seen in FIG. 10A. When torqued in the opposing direction, the structure loosens and contracts in diameter, as seen in FIG. 10B. The torque characteristics of structure S may be utilized to expand and contract an embolic filter, as well as to capture emboli disposed within the filter.

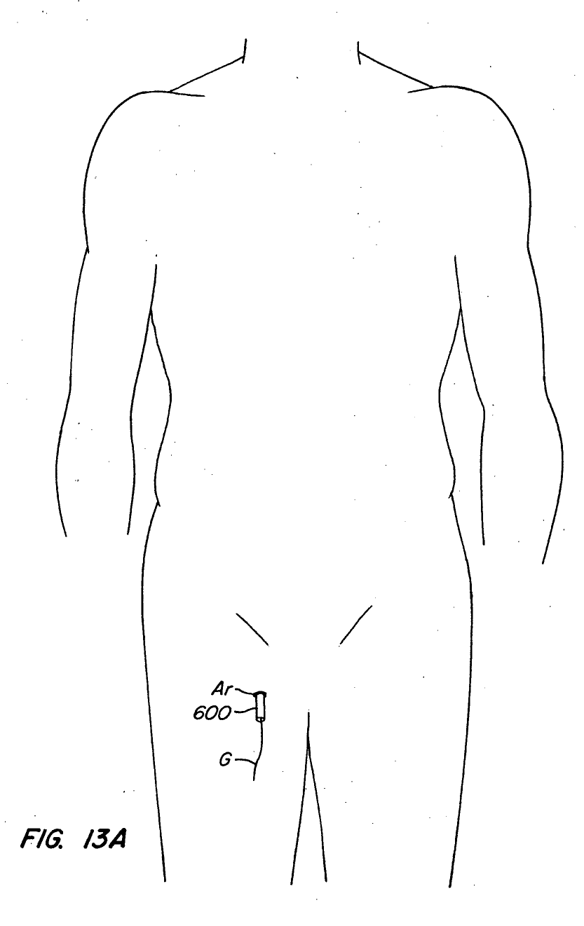

As shown in FIG. 11, filter 520 may comprise multiple longitudinal supports wound in long spirals. The supports may increase hoop strength. They may also help maintain a desired length of the filter.

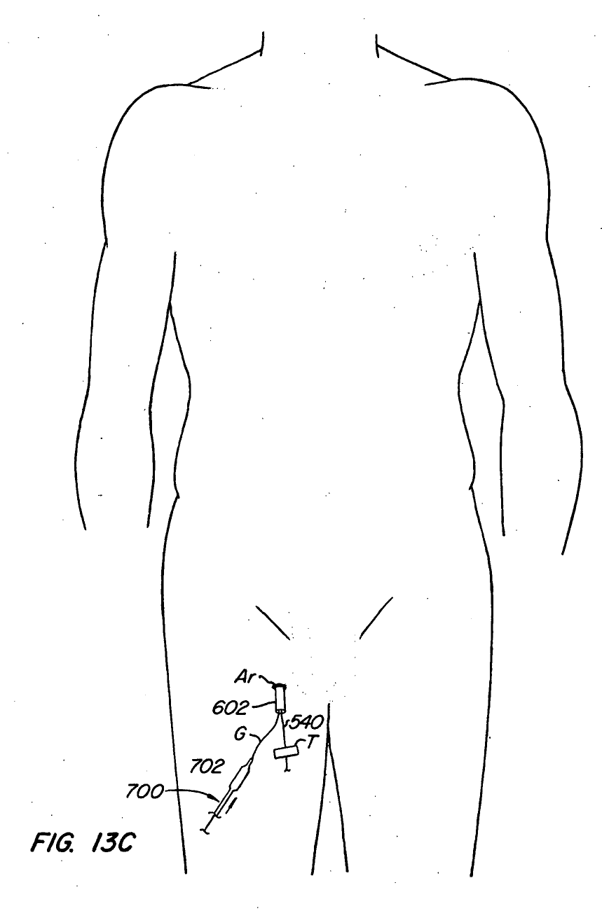

FIGS. 12 illustrate alternative deployment and retrieval methods for filter 520. In FIG. 12A, the proximal end of filter 520 is attached to the distal end of sheath 510. The filter and sheath may be advanced and withdrawn together with the filter conforming to the patient's anatomy as it is it repositioned. Alternatively, an additional over-sheath may be provided for collapsing the filter to a reduced delivery and retrieval configuration.

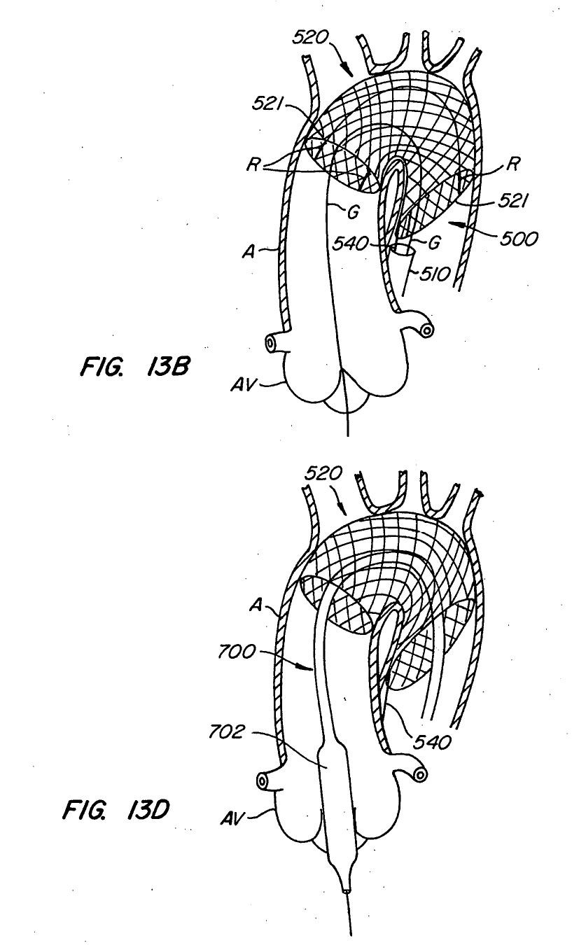

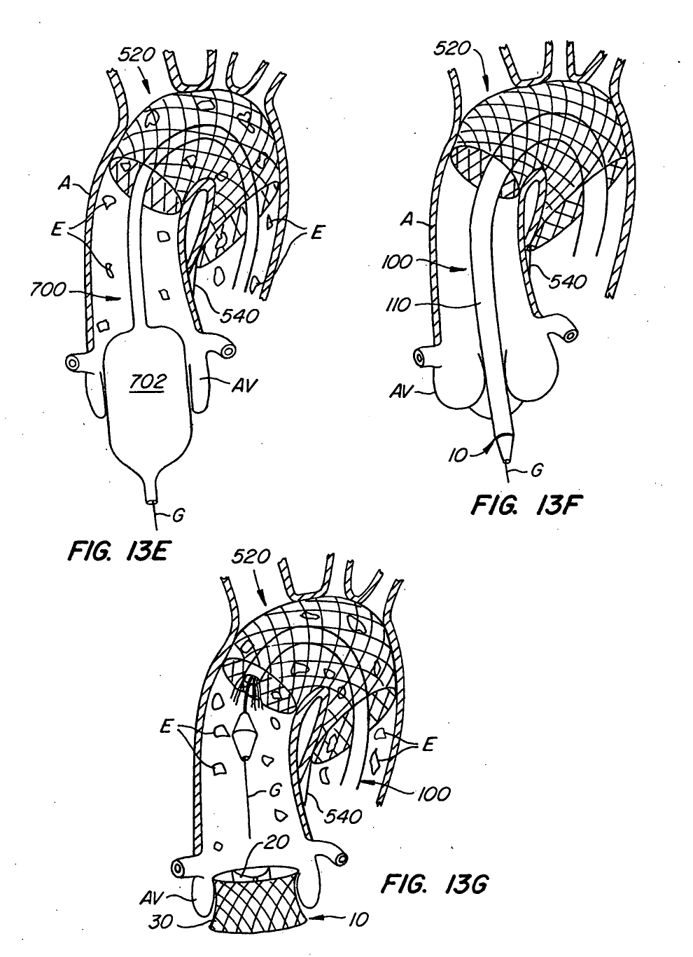

As seen in FIG. 12B, filter 520 alternatively may be collapsed within sheath 510 during delivery and retrieval, e.g. via a pullwire coupled to a proximal end of the filter (see FIGS. 13). As seen in FIG. 12C, embolic protection system 500 optionally may comprise pullwire 540 attached to the distal outlet of filter 520. By keeping the wire taut during retrieval of filter 520, it is expected that a risk of snagging, or otherwise hanging up, filter 520 on sheath 510 will be reduced.

Prior to implantation of a replacement valve, such as those described above, it may be desirable to perform a valvuloplasty on the diseased valve by inserting a balloon into the valve and expanding it, e.g., using saline mixed with a contrast agent. In addition to preparing the valve site for implantation, fluoroscopic viewing of the valvuloplasty will help determine the appropriate size of replacement valve implant to use. During valvuloplasty, embolic protection, e.g., utilizing any of the embolic filters described previously, may be provided.

Referring now to FIGS. 13, a method of replacing a patient's diseased aortic valve utilizing replacement valve apparatus 10 and delivery system 100, in combination with a diverter embodiment of embolic protection system 500, is described. Although a retrograde approach via the femoral artery illustratively is utilized, it should be understood that alternative approaches may be utilized, including, but not limited to, radial or carotid approaches, as well as trans-septal antegrade venous approaches.

As seen in FIG. 13A, arteriotomy puncture site Ar is formed, and introducer sheath 600 is advanced in a minimally invasive fashion into the patient's femoral artery. The introducer preferably initially comprises a relatively small sheath, for example, an introducer sheath on the order of about 6 Fr-compatible. Guidewire G is advanced through the introducer sheath into the femoral artery, and is then further advanced through the patient's aorta and across the patient's diseased aortic valve.

Additionally, imaging may be performed to determine whether the patient is a candidate for valvuloplasty and/or endovascular valve replacement. For example, angiographic imaging, per se known, may be performed via an angiography catheter (not shown) advanced from a femoral, radial, or other appropriate entry site. The angiography catheter may, for example, have a profile on the order of about 5 Fr to 8 Fr, although any alternative size may be used.

If it is determined that the patient is not a candidate for valvuloplasty and/or endovascular valve replacement, the guidewire and introducer sheath (as well as any imaging apparatus, e.g., the angiography catheter) may be removed from the patient, and the arteriotomy site may be sealed. If it is determined that the patient is a candidate, the arteriotomy site may be expanded, and, upon removal of any imaging apparatus, introducer sheath 600 may be exchanged with a larger introducer sheath 602 (see FIG. 13C), for example, an introducer sheath on the order of about 14 Fr compatible, to facilitate endovascular valvuloplasty and/or valve replacement.

As seen in FIG. 13B, embolic protection system 500 then may be advanced over guidewire G to the vicinity of the patient's diseased valve. Sheath 510 may be retracted relative to diverter filter 520, such that the diverter filter, which preferably comprises a self-expanding wire braid, expands into contact with the wall of aorta A downstream of aortic valve AV. Sheath 510 of embolic protection system 500 then may be removed from the patient.

Filter 520 is configured to divert emboli, generated during endovascular treatment of valve AV, away from the patient's cerebral vasculature. The filter illustratively comprises optional proximal and distal interfaces 521 of enlarged diameter that contact the wall of aorta A, while a central section of the filter disposed between the interfaces moves freely or ‘floats’ without engaging the aorta. This may reduce friction during deployment and/or retrieval of the filter, and may also reduce damage caused by the filter to the wall of the aorta. Filter 520 alternatively may contact aorta A along its length, as in FIGS. 13D-13G. Filter 520 also optionally may comprise internal rails R that may be used to guide endovascular treatment tools through the filter. Filter 520 illustratively is coupled proximally to pullwire 540, which extends from the proximal end of the filter to the exterior of the patient. Pullwire 540 allows a medical practitioner to maneuver filter 520, as desired.

As seen in FIG. 13C, upon removal of sheath 510 from the patient, guidewire G and pullwire 540 extend through introducer sheath 602. Advantageously, with filter 520 positioned as desired within the patient's aorta and with slack removed from pullwire 540, the filter may be maintained at the desired position by reversibly maintaining the position of pullwire 540, e.g., by reversibly attaching the pullwire to the exterior of the patient via surgical tape T. In this manner, a medical practitioner may properly position diverter filter 520, then leave it in the desired position without requiring significant manipulation or monitoring during endovascular treatment of the patient's diseased aortic valve AV. The open proximal end of diverter filter 520 allows additional endovascular tools, such as valvuloplasty catheter 700 and/or replacement valve apparatus 10 disposed within delivery system 100, to be advanced through the diverter.

In FIGS. 13C and 13D, optional valvuloplasty catheter 700, having expandable balloon 702, is advanced over guidewire G and through introducer sheath 602 into the patient's vasculature. Catheter 700 preferably comprises a delivery profile on the order of about 8-16 Fr, while balloon 702 preferably comprises an expanded diameter on the order of about 18 mm to 30 mm, more preferably about 20 mm to 23 mm. Proper sizing of balloon 702 optionally may be determined, for example, via angiographic imaging of aortic valve AV.

Balloon 702 is endovascularly advanced through aorta A and diverter filter 520 across diseased aortic valve AV. Diverter filter 520 advantageously guides catheter 700 past the arterial branches of aorta A as the catheter passes through the filter. In this manner, filter 520 facilitates proper placement of balloon 702, while reducing a risk of injury to the arterial branches.

In FIG. 13E, once positioned across the aortic valve, balloon 702 is expanded to break up or otherwise crack calcification and/or lesion(s) along the valve. Expansion may, for example, be achieved using saline mixed with a contrast agent. In addition to preparing the valve site for implantation, fluoroscopic viewing of the contrast agent and the valvuloplasty may help determine the appropriate size of replacement valve apparatus 10 to use. Balloon 702 is then deflated, and valvuloplasty catheter 700 is removed from the patient. Emboli E generated during valvuloplasty travel downstream through aorta A, where they are diverted by filter 520 away from the patient's cerebral vasculature.

Optionally, multiple catheters 700 may be provided and used sequentially to perform valvuloplasty. Alternatively or additionally, multiple catheters 700 may be used in parallel (e.g., via a ‘kissing balloon’ technique). The multiple catheters may comprise balloons 702 of the same size or of different sizes.

After optionally performing valvuloplasty, aortic valve AV may once again be imaged, e.g. via fluoroscopy and angiography, to determine whether the patient is a candidate for endovascular valve replacement. If it is determined that the patient is not a candidate, embolic protection system 500, as well as guidewire G and introducer sheath 602, may be removed from the patient, and arteriotomy site AR may be sealed. A suction catheter optionally may be positioned within filter 520 prior to retrieval of the filter to ‘vacuum out’ any emboli caught therein.

In order to collapse filter 520 for retrieval, sheath 510 of embolic protection system 500 optionally may be re-advanced through introducer 602 and over pullwire 540 (optionally, also over guidewire G) to contact a proximal region of the filter (see FIGS. 12). The tapered proximal region may function as collapse element that facilitates sheathing of filter 520 for delivery and/or retrieval, e.g., by distributing forces applied to the filter by sheath 510 along a greater longitudinal length of the filter, as compared, for example, to embodiments of the filter that are not proximally tapered. Additional and alternative collapse elements may be provided with filter 520 or with sheath 510. The collapse element may collapse the filter, e.g., by collapsing the filter braid.

Filter 520 alternatively may be retrieved by proximally retracting pullwire 540 without collapsing the filter within a retrieval sheath, thereby proximally retracting filter 520 directly through the patient's vasculature. As yet another alternative, a specialized retrieval sheath, e.g., a sheath of larger or smaller profile than sheath 510, may be utilized. The retrieval sheath optionally may comprise a distally enlarged lumen to accommodate the collapsed filter.

In FIG. 13F, if it is determined that the patient is a candidate for endovascular valve replacement, delivery system 100, having replacement valve apparatus 10 disposed therein in a collapsed delivery configuration, may be endovascularly advanced over guidewire G through the introducer sheath, through filter 520 and across the patient's aortic valve AV. As during advancement of balloon catheter 700, diverter filter 520 advantageously guides delivery system 100 past arterial branches of aorta A, while the delivery system is advanced through the filter. In this manner, filter 520 facilitates proper positioning of apparatus 10, while protecting the aortic side branches from injury.

As it is expected that delivery system 100 may have a delivery profile on the order of about 18-21 Fr, preferably about 19 Fr, introducer sheath 602 optionally may be exchanged for a larger introducer sheath in order to accommodate the delivery system. Alternatively, in order to reduce the size of arteriotomy site AR, it may be desirable to remove the introducer sheath and to advance delivery system 100 directly through the arteriotomy site without an intervening introducer sheath, such that sheath 110 of the delivery system acts as the introducer sheath. Delivery system 100 optionally may comprise a rapid-exchange lumen for advancement over guidewire G.

If introducer sheath 602 is exchanged or removed, pullwire 540 temporarily may be disconnected from the exterior of the patient, e.g., by removing tape T. The introducer sheath then optionally may be removed or exchanged, and pullwire 540 may be re-affixed to the patient. During removal and/or exchange of introducer sheath 602 (i.e., while pullwire 540 is not affixed to the patient), a medical practitioner preferably grasps pullwire 540 and maintains its position relative to arteriotomy site AR, thereby maintaining the position of filter 520 deployed within the patient.

In FIG. 13G, once replacement valve apparatus 10 has been properly positioned across the patient's diseased aortic valve AV, sheath 110 of delivery system 100 may be retracted, and apparatus 10 may be deployed as described previously, thereby endovascularly replacing the patient's diseased valve. Emboli E generated during deployment of apparatus 10 are diverted away from the patient's carotid arteries and cerebral vasculature by filter 520. Delivery system 100 then may be removed from the patient.

Filter 520 optionally may be vacuumed out via a suction catheter, e.g., suction drawn through sheath 110. Filter 520 and guidewire G then may be removed from the patient as discussed previously, and arteriotomy site AR may be sealed to complete the procedure. Guidewire G may retrieved and removed before, during or after retrieval and removal of filter 520. Retrieval and removal of the filter may comprise reintroduction of sheath 510 (e.g., over pullwire 540 and directly through the arteriotomy site, through an introducer sheath or through sheath 110 of delivery system 100) and collapse of filter 520 within the sheath. Alternatively, removal of filter 520 may comprise retraction of pullwire 540 without collapse of the filter in an intervening retrieval sheath. Sealing of the arteriotomy site may comprise any known sealing method, including, but not limited to, application of pressure, introduction of sealants, suturing, clipping and/or placement of a collagen plug.

In FIGS. 13, although diversion and/or filtering of emboli illustratively has been conducted during both valvuloplasty and endovascular deployment of replacement valve apparatus, it should be understood that such diversion/filtering alternatively may be performed only during valvuloplasty or only during endovascular valve replacement. Furthermore, it should be understood that embolic protection may be provided during deployment of any endovascular replacement valve apparatus and is not limited to deployment of the specific embodiments of such apparatus described herein.

1. Apparatus for protecting against embolization during endovascular replacement of a patient's heart valve, the apparatus comprising:
a replacement valve configured for endovascular delivery and deployment; and
an embolic filter configured for disposal downstream of the replacement valve during deployment of the valve to divert emboli away from the patient's cerebral vasculature without capturing the emboli within the filter.

2. The apparatus of claim 1, wherein the embolic filter is coupled to the replacement valve.

3. The apparatus of claim 1, wherein the embolic filter is decoupled from the replacement valve.

4. The apparatus of claim 1, wherein the embolic filter is configured for expansion from a collapsed delivery configuration to an expanded deployed configuration.

5. The apparatus of claim 4, wherein the embolic filter is configured to contact a patient's aorta and form a circumferential seal against the aorta in the deployed configuration.

6. The apparatus of claim 4, wherein the embolic filter is configured for endovascular delivery in the collapsed delivery configuration.

7. The apparatus of claim 1, wherein the replacement valve is configured for endovascular delivery through the embolic filter.

8. The apparatus of claim 1 further comprising a suction element configured to aspirate diverted emboli from the patient's bloodstream.

9. The apparatus of claim 1, wherein the embolic filter is fabricated from an expandable wire braid or mesh.

10. The apparatus of claim 1, wherein the embolic filter comprises a spiral-wound structure.

11. The apparatus of claim 10, wherein the spiral-wound structure is configured to expand when torqued in a first direction and to contract when torqued in an opposite direction.

12. The apparatus of claim 1, wherein the embolic filter comprises a permeable membrane having a specified porosity.

13. The apparatus of claim 12, wherein the specified porosity comprises pores less than about 100 μm in diameter.

14. The apparatus of claim 12, wherein the permeable membrane comprises a varying porosity.

15. The apparatus of claim 1 further comprising an expandable balloon for performing valvuloplasty,
wherein the embolic filter is configured to divert emboli generated during valvuloplasty.

16. The apparatus of claim 1, wherein the embolic filter comprises at least one measuring element for determining distances within the patient.

17. The apparatus of claim 16, wherein the measuring element is configured to provide a center-axis distance between the patient's heart valve and a desired location within the patient's aorta.

18. The apparatus of claim 4, wherein the embolic filter comprises a curved profile in the deployed configuration.

19. The apparatus of claim 4, wherein the embolic filter is configured for collapse from the expanded deployed configuration to a collapsed retrieval configuration.

20. The apparatus of claim 19 further comprising a collapse element adapted to facilitate collapse and retrieval of the filter from the patient.

21. The apparatus of claim 20, wherein the embolic filter comprises the collapse element.

22. The apparatus of claim 21, wherein the collapse element comprises a tapered opening disposed at a proximal end of the embolic filter.

23. The apparatus of claim 20, wherein the collapse element comprises a retrieval sheath advanceable over the filter.

24. The apparatus of claim 5, wherein the embolic filter further comprises proximal and distal interfaces, and
wherein the embolic filter is configured to contact the patient's aorta only along the proximal and distal interfaces.

25. The apparatus of claim 7, wherein the embolic filter is configured to guide a catheter from a proximal end of the filter to a distal end of the filter.

26. The apparatus of claim 9, wherein the expandable wire braid or mesh further comprises a Nitinol wire braid or mesh.

27. A method for protecting a patient against embolization during endovascular replacement of the patient's heart valve, the method comprising:
endovascularly delivering a replacement valve to a vicinity of the patient's heart valve;
endovascularly deploying an embolic filter downstream of the heart valve; and
endovascularly deploying the replacement valve; and
diverting emboli away from the patient's cerebral vasculature with the filter without capturing the diverted emboli within the filter.

28. The method of claim 27 further comprising aspirating the emboli via suction.

29. The method of claim 27, wherein endovascularly deploying the replacement valve further comprises displacing the patient's heart valve with the replacement valve.

30. The method of claim 27 further comprising diverting emboli with the embolic filter.

31. The method of claim 30, wherein diverting emboli further comprises diverting the emboli away from the patient's cerebral vasculature.

32. The method of claim 27 further comprising removing the embolic filter from the patient after deployment of the replacement valve.

33. The method of claim 27, wherein protecting a patient against embolization during endovascular replacement of the patient's heart valve further comprises protecting the patient during endovascular replacement of the patient's aortic valve, and
wherein endovascularly delivering the replacement valve further comprises endovascularly delivering the replacement valve along a retrograde approach.

34. The method of claim 33, wherein endovascularly deploying an embolic filter downstream of the heart valve further comprises endovascularly deploying the embolic filter in the patient's aorta.

35. The method of claim 27 further comprising:
endovascularly delivering an expandable balloon to the vicinity of the heart valve; and
performing valvuloplasty with the expandable balloon.

36. Apparatus for protecting against embolization during endovascular replacement of a patient's heart valve, the apparatus comprising:
a delivery catheter having an expandable replacement valve disposed therein; and
an embolic filter advanceable along the delivery catheter for diverting emboli released during endovascular deployment of the replacement valve,
wherein the embolic filter is configured to divert the emboli without capturing the emboli within the filter.

37. A kit for endovascularly replacing a patient's diseased heart valve, the kit comprising:
a valvuloplasty balloon catheter;
a expandable replacement valve configured for endovascular delivery and deployment across the patient's diseased valve; and
an embolic filter configured for endovascular delivery and deployment downstream of the patient's diseased valve.

38. The kit of claim 37, wherein the filter is configured to filter or divert emboli generated during valvuloplasty or deployment of the replacement valve.

39. The kit of claim 37 further comprising a delivery system for endovascularly delivering and deploying the expandable replacement valve.

40. The kit of claim 37 further comprising a delivery system for endovascularly delivering and deploying the embolic filter.

41. A method for endovascularly replacing a patient's diseased heart valve in a protected fashion, the method comprising:
deploying an embolic filter downstream of the patient's diseased heart valve;
performing valvuloplasty on the diseased valve; and
endovascularly deploying a replacement valve across the diseased valve.

42. The method of claim 41 further comprising diverting emboli generated during valvuloplasty away from the patient's cerebral vasculature with the embolic filter.

43. The method of claim 41 further comprising diverting emboli generated during endovascular deployment of the replacement valve away from the patient's cerebral vasculature with the embolic filter.

44. The method of claim 41 further comprising maintaining a position of the embolic filter during valvuloplasty and endovascular deployment of the replacement valve.

45. The method of claim 44, wherein maintaining a position of the embolic filter further comprises maintaining a position of an elongated member that is attached to the filter and extends out of the patient.

46. The method of claim 45, wherein maintaining a position of the elongated member further comprises reversibly affixing the elongated member to an exterior of the patient.

47. The method of claim 41, wherein performing valvuloplasty further comprises endovascularly advancing a valvuloplasty catheter through the deployed embolic filter.

48. The method of claim 41, wherein endovascularly deploying a replacement valve further comprises endovascularly advancing the replacement valve through the deployed embolic filter.

49. The method of claim 41 further comprising removing the embolic filter from the patient.

50. The method of claim 41, wherein deploying an embolic filter further comprises deploying proximal and distal interfaces of the filter into contact with the patient's aorta.
